(19) United States
(12) Patent Application Publication
(10) Pub. No.: US 2025/0327016 A1
(43) Pub. Date: Oct. 23, 2025

(54) BIOREACTOR FOR ALGAL GROWTH

(71) Applicant: SEASCAPE RESTORATIONS AUSTRALIA, T/A IMMERSION GROUP, Watermans Bay, Western Australia (AU)

(72) Inventors: John STATTON, Duncraig (AU); Scott Charles ELLIOTT, St Leonards (AU)

(21) Appl. No.: 19/122,014

(22) PCT Filed: Oct. 18, 2023
(86) PCT No.: PCT/AU2023/051035

§ 371 Date: Apr. 17, 2025

(30) Foreign Application Priority Data
Oct. 18, 2022 (AU) 2022903067

(51) Int. Cl. C12N 1/12; C12M 1/00; C12M 1/34; C12M 3/00

(52) U.S. Cl. CPC C12N 1/12; C12M 21/02; C12M 23/44; C12M 23/48; C12M 41/06; C12M 41/12; C12M 41/42

(57) ABSTRACT

A photobioreactor (PBR) cell comprising a riser portion in the form of a tube; a downcomer portion in the form of a tube; a means of joining the riser portion and the downcomer portion to allow liquid flow; and an aeration means.

### RELATED APPLICATIONS

This application claims the benefit of Australian Provisional Patent Application No. 2022903067, filed on 18 Oct. 2022, which is incorporated herein by reference in its entirety.

### TECHNICAL FIELD

The present invention relates to modularisable photobioreactor cells for production of algae, methods of growing algae, and algal cultures and compositions.

### BACKGROUND ART

Livestock production, particularly ruminants, contributes to anthropogenic greenhouse gas (GHG) emissions globally. The majority of GHG emissions from livestock production are in the form of methane (CH₄), which is produced largely through enteric fermentation, and to a lesser extent manure decomposition. Enteric CH₄ emissions not only contribute to total agricultural GHG emissions but also represent an energy loss amounting to 11% of dietary energy consumption. Therefore, reducing enteric CH₄ emissions decreases the total agricultural contribution to climate change and can improve productivity through conservation of feed energy.

Mitigation of enteric CH₄ emissions via organic feed supplements derived from red seaweeds Asparagopsis taxiformis (A. taxiformis) and Asparagopsis armata (A. armata) modify the rumen environment and directly inhibit methanogenesis resulting in lower enteric CH₄ production ruminant livestock (>80% reduction). Asparagopsis spp. synthesize and store halogenated CH₄ analogues, such as bromoform and dibromochloromethane, within specialized gland cells as a natural defence mechanism.

Asparagopsis spp. have been found to reduce CH₄ more effectively compared to similar inclusions of pure bromoform in vitro likely due to multiple anti-methanogenic CH₄ analogues such as bromo—and iodo-methanes and—ethanes that work synergistically, and that methanogen species are differentially sensitive to CH₄ inhibitors.

Approaches for commercial-scale production of Asparagopsis spp. would allow for large-scale use of the seaweeds in ruminant livestock feeds. Accordingly, there is a need for a low capital cost, scalable production system, serving as a means for producing a feedstock usable as an organic feedstock for reducing GHG emissions and improving productivity of ruminant livestock. Improved culturing methods and an improved feedstock would also be desirable. Typical open ocean farming of Asparagopsis gives an annual seaweed yield of between 1-3.8 tonnes dry weight per hectare using current practices (Agrifutures 2022). However, many seas are not suitable for growing Asparagopsis seaweeds. One sustainable alternative is the use of coastal non-agricultural lands for growing seaweeds. The coastal zone can provide many thousands to tens of thousands of hectares for onshore seaweed farming. Onshore seaweed farming is, however, very different from open sea cultivation and is fraught with different challenges. For example, attempts have been made to use onshore tanks, ponds or enclosed indoor photobioreactors modules. These methods were found to be not cost effective at scale.

There is a need for new methods and apparatus for the growth of algae; or at least the provision of methods and apparatus to complement the previously known methods and apparatus for the growth of algae. The present invention seeks to provide one or more of improved or alternative methods and apparatus for the growth of algae and improved or alternative algae cultures and compositions.

The previous discussion of the background art is intended to facilitate an understanding of the present invention only. The discussion is not an acknowledgement or admission that any of the material referred to is or was part of the common general knowledge as at the priority date of the application.

### SUMMARY OF DISCLOSURE

The present invention provides a photobioreactor (PBR) cell comprising:
a) a riser portion in the form of a tube;
b) a downcomer portion in the form of a tube;
c) a means of joining the riser portion and the downcomer portion to allow liquid flow;
d) an aeration means.

Preferably the PBR cell is used for or adapted to be used for the growth of an algae species, more preferably a macroalgae. Preferably the macroalgae is an Asparagopsis spp.

Preferably the tubes of the PBR cells are made from UV-resistant, low density plastic film, with a diameter of from 75 to 300 mm, and/or a length of from 500 to 3000 mm. Preferably the total working volume of each PBR cell is from 1.5 to 215 litres.

Preferably the temperature of the PBR cells is maintained from 12° C. to 28° C. Preferably the aeration rate of the PBR cells is from 1 to 70 L/min. Preferably the PBR cells of the present invention are exposed to a light intensity of from 100 to 1500 μmols m⁻²·s⁻¹ and a photoperiod of from 8:16 to 14:10 light: dark cycle.

The present invention further provides a method of growing algae comprising the steps of:
i. inoculating a photobioreactor (PBR) cell with algal cells, wherein the PBR cell comprises:
   a. a riser portion in the form of a tube;
   b. a downcomer portion in the form of a tube;
   c. a means of joining the riser portion and the downcomer portion to allow liquid flow;
   d. an aeration means;
ii. maintaining the PBR cells in an aqueous culture medium under conditions of light and aeration that suit the growth of the algal cells.

The present invention further provides a method of growing algae comprising the steps of:
i. providing algal cells in aqueous culture medium having a biomass density of greater than 0.1 g/L and an average fragment diameter;
ii. removing algal biomass from the culture medium to produce a culture of retained algal cells having:
   a. a biomass density of at least 0.1 g/L and
   b. an average fragment diameter less than the average fragment diameter of the algal cells in aqueous culture medium prior to removing algal biomass; and
iii. maintaining the culture of retained algal cells in aqueous culture medium under conditions of light and aeration that suit the growth of the algal cells.

The present invention provides a culture of algal cells produced using a method of growing algae using a PBR cell as herein described. Preferably, said culture of algal cells has an average fragment diameter.

The present invention further provides a culture of retained algal cells produced using a method of growing algae using a PBR cell as herein described. The culture of retained algal cells is a culture of algal cells grown using a PBR cell as described above, from which algal biomass has been removed. Preferably, the culture of retained algal cells has an average fragment diameter of less than the average fragment diameter of the culture of algal cells immediately prior to removal of the algal biomass.

The present invention further provides a composition comprising algal biomass. Algal biomass is biomass produced in a culture of algal cells by a method of growing algae using a PBR cell as herein described. Preferably, said algal biomass has an average fragment diameter.

The present invention further provides a composition comprising removed algal biomass. Removed algal biomass is biomass removed from algal biomass in a culture as described above. Preferably, said removed algal biomass has an average fragment diameter greater than the average fragment diameter of the algal biomass from which the removed algal biomass was removed. Preferably, the removed algal biomass has at least 60%, preferably 70%, 80% or 90% of the algal biomass with a fragment diameter of at least 2 mm.

The present invention further provides a composition comprising retained algal biomass. Retained algal biomass is biomass retained in a culture of algal cells as described above, after removal of removed algal biomass. Preferably, said retained algal biomass has an average fragment diameter less than the average fragment diameter of the algal biomass from which the removed algal biomass was removed. Preferably, the retained algal biomass has at least 60%, preferably 70%, 80% or 90% of the algal biomass with a fragment diameter of less than 2 mm.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description as set out above. The description will be made with reference to the accompanying drawings in which:

FIG. 1 is a diagram of an example photobioreactor cell in accordance with the present invention.

FIG. 2 is a diagram of an example photobioreactor cell (FIG. 2A) and photobioreactor cells connected in series (FIG. 2B) in accordance with the present invention.

FIG. 3 is an image of Asparagopsis taxiformis tetrasporophyte cultures, showing that tetrasporophytes can self-fragment and continue to grow so that a continuous harvest process is required to maintain culture density. FIG. 2(a) shows a dense culture (5 g/L) with a range of tetrasporophyte sizes, reaching a harvestable size of >8 mm in diameter. Removal of airlift reveals the culture is also made up of FIG. 2(b) medium (3-7 mm) fragments and FIG. 2(c) small (<3 mm) fragments.

FIG. 4 is an image of Asparagopsis taxiformis tetrasporophytes cultured under optimized operations within interconnected photobioreactor cells (10 cells), demonstrating a 30-day production cycle to harvestable size.

### DETAILED DESCRIPTION

To provide a new approach to the problems associated with onshore growth of algae, the present invention in one aspect is aimed at providing a system for cost-effective, large-scale onshore algal farming, and in other aspects is aimed at providing improved algae growing methods, cultures and compositions.

The device of the present invention generally comprises a photobioreactor cell (PBR cell) that can be used in a series setup to produce a low capital cost, modularizable photobioreactor system (PBR system). The PBR system can also optionally offer advantages such as the ability to control the photoperiod of light provided to the algae, which may be used to induce sporulation and/or permit continuous harvesting to control biomass.

The PBR cells and PBR system of the present invention were designed as a modular, linearly scalable, and resilient system that may operate with low land and water footprints. It may give an increase in renewable algal-based feedstock production compared to the conventional sea-based and other on-shore tank-based algal growth systems.

Methods of the present invention generally comprise growing algae in an aqueous culture medium using PBR cell(s). The methods may include the step of removal of algal biomass from the aqueous culture medium. Preferably, the removed biomass has an average fragment diameter greater than the average fragment diameter of the culture prior to removal of the algal biomass. Removal of the biomass preferably results in the production of a culture having average fragment diameter less than the average fragment diameter of the culture prior to removing algal biomass.

The methods of the present invention are designed to potentially allow the continuous culturing of algae. The continuously cultured algae may allow for the continuous harvesting of algal biomass for downstream uses, in particular as a feedstock for ruminant feed.

Thus, cultures and compositions of the present invention generally comprise:
cultures of algal cells with an average fragment diameter;
cultures from which algal biomass has been removed, such retained algal cell cultures having an average fragment diameter less than the average fragment diameter of the algal culture prior to the removal of algal biomass;
compositions comprising algal biomass with an average algal fragment diameter;
compositions comprising removed algal biomass removed from a culture of algal cells, said removed algal biomass compositions having an average fragment diameter greater than the average fragment diameter of the algal culture prior to the removal of algal biomass; and
compositions comprising retained algal biomass, said retained algal biomass being the algal biomass retained after removal of the removed algal biomass, said retained algal biomass compositions having an average fragment diameter less than the average fragment diameter of the algal culture prior to removing algal biomass.

### Algae

In the present invention, the terms “seaweed” and “algae” are used interchangeably. Algae are simple, non-flowering, and typically eukaryotic photosynthetic aquatic organisms. Algae contain chlorophyll but lack true stems, roots, leaves, and vascular tissue. The algae may be a macroalgae or a microalgae.

A microalgae is unicellular algae throughout its lifecycle while a macroalgae has at least one multicellular stage during its life cycle When in association with an aqueous culture medium, the algae of the present invention may be referred to herein as “algal cells”. When not in association with an aqueous culture medium, the algae of the present invention may be referred to herein as “algal biomass”.

Algae suitable for use in the present invention are preferably macroalgae. These macroalgae include green, brown and red algae. Brown and red algae are preferred because they typically require weaker light intensity than green algae to grow, which may reduce the electrical cost for onshore farming using artificial lighting. Preferably, the macroalgae is red algae. Red algae is preferred because it tends to produce extracellular material, including cell-wall polysaccharides, which may result in an improved ruminant feed.

Algal cells in aqueous culture medium may form multi-cellular clusters, especially under growth conditions. These clusters may be referred to as “biomass fragments” or simply “fragments”. Fragments may range in size from a diameter of less than 2 mm to greater than 8 mm. A biomass fragment having a diameter of less than 2 mm may be referred to as “very small”, a biomass fragment having a diameter of from 2 mm to 4 mm may be referred to as “small”, a biomass fragment having a diameter of from 4 mm to 6 mm may be referred to as “medium”, while a biomass fragment having a diameter of greater than 6 mm may be referred to as “large”. The term “diameter” in the context of biomass fragments does not limit the shape of a fragment and refers to the greatest axial dimension. Biomass fragments may form during the gametophyte or sporophyte phase. The sporophyte of an algae may be, and preferably is, a tetrasporophyte.

Preferably, the alga is of the class Florideophyceae. Florideophyceae are multicellular red algae which form biomass fragments. Preferably, the alga is of the order Bonnemaisoniales in the class Florideophyceae. Bonnemaisonialea form biomass fragments in the sporophyte phase including as tetrasporophytes. Preferably, the alga is an Asparagopsis spp. as described below. The Asparagopsis spp. may be, and is preferably, Asparagopsis taxiformis (A. taxiformis) and/or Asparagopsis armata (A. armata). Asparagopsis spp. are macroalgae, although the algae may be in the form of very small (microscopic) fragments, for example after maceration prior to inoculation or during early growth. The macroalgal Asparagopsis fragments may initially be as small as one or a few cells and may be microscopic; however, the cells will undergo substantial cell divisional and form clearly visible macroalgal fragments after a day or more growth.

### Asparagopsis

A. taxiformis has a wide climatic range but typically proliferates in warm temperate to tropical climates whereas A. armata is typically proliferates in cool temperate climates. Both species have a diplo-haplontic life-cycle with three morphologically distinct stages (two macroscopic and one microscopic stage); gametophytes (macroscopic), carposporophytes (microscopic) and tetrasporophytes (macroscopic). At the gametophytic stage, both species share similar characteristics but are morphologically distinct from one another. Both species have rhizoids that give rise to several erect, polysiphonous stems. These ramify repeatedly into trisiphonous ramuli, defining the thallus. The gametophytes produce male gametes on antheridia and femail gametes on carpogonia. In contrast, A. armata has spinose branches (harpoon-like serrated appendage), highly elongate erect branches and a sprawling habit in which the spines entangle among other benthic organism and artificial structures. A. taxiformis has a more compact rhizoidal system, lacks spiny branches, and forms more patchy tufts.

The carposporophyte, is a microscopically sized life-stage and remains attached to the female gametophyte. Carposporophytes produce carpospores, which are released in the water column and, upon settlement, develop into tetrasporophytes called the “Falkenbergia-stage” (A. taxiformis—F. hillebrandii; A. armata—F. rufolanosa). These look like red pom-poms as they develop by ramifying trisiphonal, branching filaments. The Falkenbergia-stage were thought to be morphologically identical, though later found to have different sizes of the terminal cells between the two species when maintained in culture. Tetrasporophytes can produce tetraspores via asexual reproduction (meiosis). Tetraspores, also released into the water column can settle on substratum and develop into gametophytes.

Both species' gametophyte and tetrasporophyte life-stages are sources of halogenated compounds, with important antifungal and antibiotic activity. The tetrasporophyte stage tends to have more halogenated compounds per unit biomass than the gametophyte stage due to less structural biomass. The terasporophyte life-stage is the focal life-stage for growth using the PBR cells of the present invention.

The commercial demand for these two species is due not only to their inherent ability to produce biologically active metabolites (e.g. bromoform as well as small quantities of other bromine, chlorine and iodine-containing methanes, ethanes, ethanols, acetaldehydes, acetones, 2-acetoxypropanes, propens, epoxypropanes, acroleins and butenones), but also to partition and store these compounds in specialized storage or gland cells to prevent autotoxicity. In addition to displaying powerful anti-methanogenic uses, the onshore cultivation of the Asparagopsis may represent a significant source of other bioactive compounds responsible for antioxidant and cytotoxic activity in pharmaceutical and veterinary settings.

The present invention provides the ability to commercially produce and harvest tetrasporophytes of A. taxiformis and/or A. armata using the PBR cells, PBR systems and methods described herein. The invention further provides access to organically produced metabolites, such as cell-wall polysaccharides, which may result in an improved ruminant feed.

Preferably the tetrasporophyte cultures of Asparagopsis spp. are maintained at a culture density in the PBR cells of from 1.0 to 9.0 g/L, more preferable from 2.0 to 7.0 g/L as described below.

### Photobioreactor Apparatus

Each PBR cell is comprised of two connected vertical plastic film tubes, with one plastic film tube acting as the riser portion of the PBR cell and the second plastic film tube as the downcomer portion of the PBR cell alternately.

Two or more PBR cells can be interconnected in series to produce a PBR system.

The present invention thus provides a photobioreactor cell comprising:
a) a riser portion in the form of a tube;
b) a downcomer portion in the form of a tube;
c) a means of joining the riser portion and the downcomer portion to allow liquid flow;
d) an aeration means.

### Reactor Tubing

Preferably the tubes of the PBR cells are made from plastic film. Thus, the PBR cells preferably comprise plastic film tubes. Preferably, the plastic film tubes are clear or have minimal opacity. The percentage of transmitted light that is scattered by the plastic film tubes is preferably from 1.3% to 27.5%. For example, the percentage of transmitted light that is scattered may be in a range with an upper limit of 27.5%, 25%, 22.5%, 20%, 17.5%, 15, %, 12.5%, 10%, 7.5%, 5%, 2.5%, 2.0%, or 1.5% and/or a lower limit of 1.3%, 1.5%, 2.0%, 2.5%, 5.0%, 7.5%, 10%, 12.5%, 15, %, 17.5%, 20%, 22.5%, or 25%.

The advantage of plastic film tubes over, for example, glass tubes, is that they are cheaper, and less prone to breakage. They are also lighter and thus easier and cheaper to transport and setup, particularly if the PBR system is being established in more remote locations.

Preferably the plastic film of the plastic film tubes is UV-resistant plastic film.

Preferably the plastic film of the plastic film tubes is low-density plastic film. Preferably the plastic film has a density of from 50 to 150 microns, preferable 100 microns.

Preferably the plastic film of the plastic film tubes is thin plastic film. Preferably the plastic film has a thickness of from 0.05 mm to 0.15 mm. More preferably the plastic film has a thickness of from 0.1 mm to 0.15 mm, for example about 0.1 mm.

Preferably the plastic film of the plastic film tubes is polyethylene (LDPE) plastic film. The tubes can also be constructed of other amorphous plastics including polyvinylchloride (transparent PVC), polycarbonate (PC), and acrylic.

Optionally the tubes may be made from glass (i.e. the term “plastic film tubes” can encompass glass tubes) or high density plastic. However, these materials generally do not provide the preferable characteristics of light transfer, cost effectiveness, ease of transport and set up etc., that low-density plastic film tubes provide.

Thus, preferably the plastic film tubes of the PBR cells are made from UV-resistant, low density plastic film, such as UV-resistant, low-density polyethylene (LDPE) plastic film.

In some embodiments of the disclosure, the diameter of the plastic film tubes is from 75 to 300 mm, from 100 to 200 mm or from 125 to 175 mm. For example, the diameter of the plastic film tubes may be 75 mm, 80 mm, 90 mm, 100 mm, 125 mm, 150 mm, 200 mm, 300 mm, more preferable 150 mm. This diameter maximises volume while still allowing light to penetrate through the entire tube.

In some embodiments of the disclosure, the length of the length of the plastic film tubes is from 500 to 3000 mm, from 1000 to 3000 mm, from 2000 to 3000 mm. For example, the length of the plastic film tubes may be 500 mm, 750 mm, 1000 mm, 1200 mm, 1500 mm, 2000 mm, 2500 mm, or 3000 mm in length, more preferable 2500 mm. This length allows the aeration to be provided at such a rate that tetrasporophytes can reach medium to large size and disperse throughout the whole volume of at least the airlift cell of the PBR cell. If the PBR cell is a vertical setup, the tube length is approximately the height of the PBR cell.

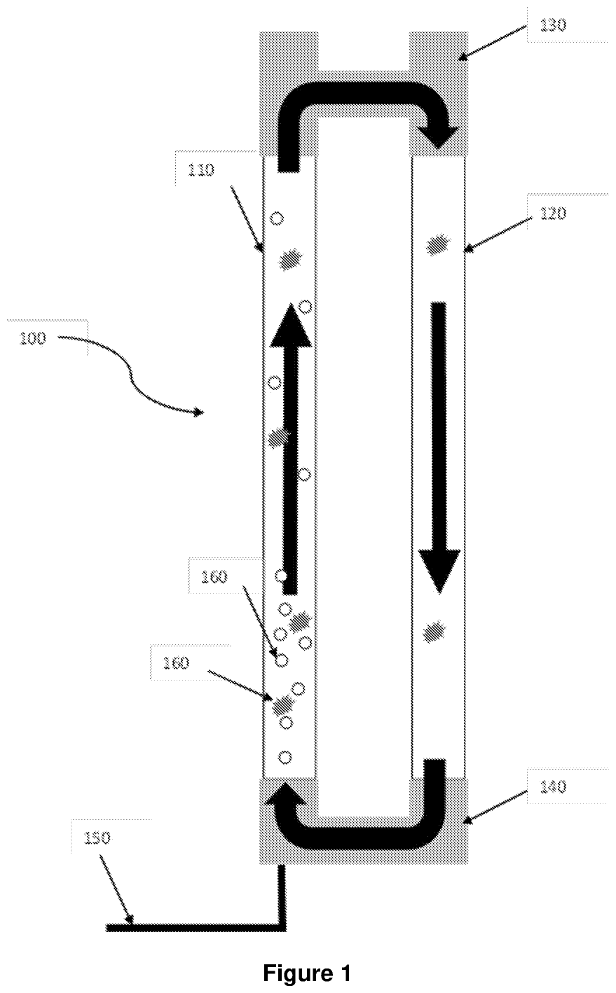

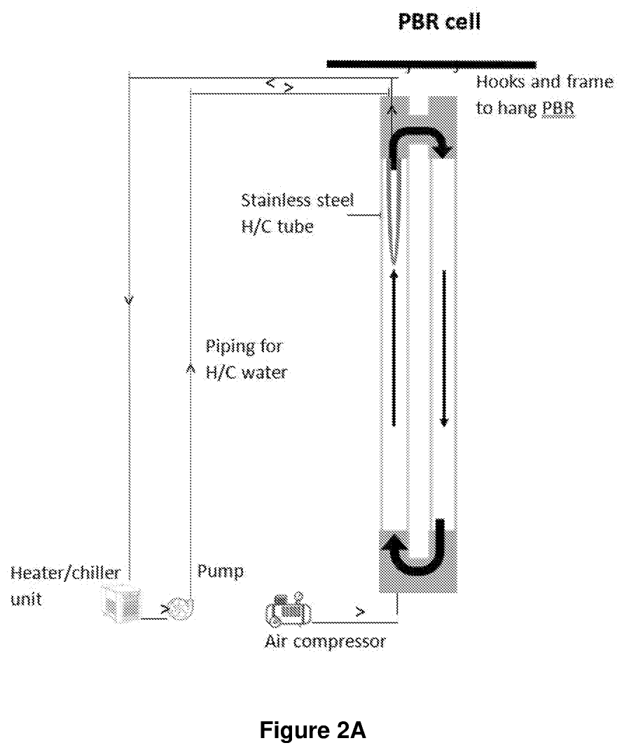

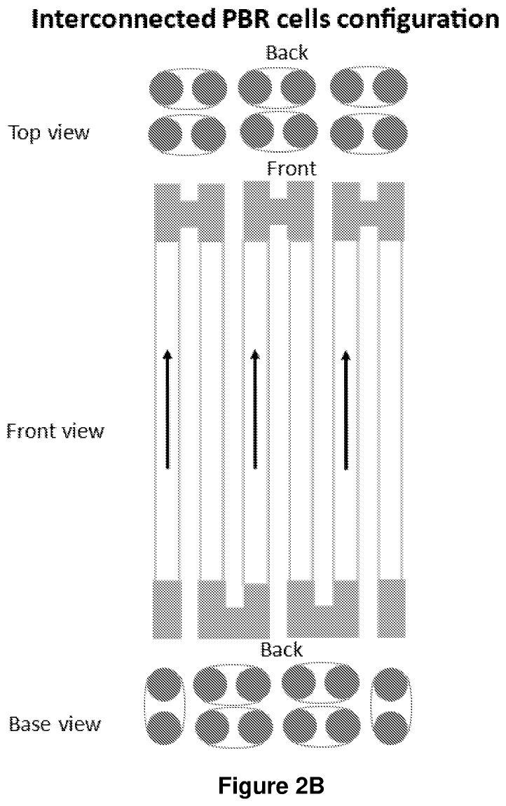

In some embodiments of the disclosure, the plastic film tubes are interconnected with U-connectors and H-connectors (see, for example, FIGS. 1 and 2). Preferably the plastic film tubes joined by connectors to form a PBR cell are arranged in a vertical setup. A vertical setup minimises the amount of area the PBR cell or PBS system occupies. In this embodiment the U-connectors are bottom U-connectors, and the H-connectors are top H-connectors. Alternatively, the PBR cell may be provided in a horizontal setup, with the U-connectors and H-connectors being provided at opposite ends of the plastic film tubes. A horizontal setup maximises the area exposed to natural lighting from above.

Preferably the U-connectors and H-connectors are made from polyvinylchloride, acrylic or low-density polyethylene (LDPE). In some embodiments of the disclosure, the U-Connectors and H-connectors are constructed from transparent acrylic material.

In some embodiments of the disclosure, the plastic film tubes of the riser portion and downcomer portion are extruded in a single unit with a U-shaped bend that serves the same purpose as the U-connector.

Preferably the U-connectors and H-connectors are clamped to the plastic film tubes via a coupling (for example a rubber coupling) and a ring clamp (for example a stainless-steel ring clamp, preferably a 316 stainless-steel ring clamp) that ensures no slippage of the plastic film tubes from the U-connector or H-connector and provides a water-tight seal. In some embodiments of the disclosure, the U-Connectors and H-connectors are constructed from transparent acrylic material. Alternatively, the U-Connectors and H-connectors may be attached to the plastic film tubes via a turn-screw fastener, such as a plastic turn-screw fastener.

In some embodiments the H-connectors are open at the top to allow off-gassing from the air of the aeration means. In some embodiments the H-connectors have a valve that prevents evaporative loss of water but allows off-gassing.

The alternating riser portion and downcomer portion can form a single PBR cell or may be interconnected as a PBR system. In a PBR system, each PBR cells' riser portion and downcomer portion forms a vertical loop, with the vertical riser portion and downcomer portion spaced 50 mm to 300 mm apart (preferable about 150 mm) occupying a total land footprint of from 0.075 m² to 0.27 m².

Preferably the total working volume of each PBR cell is from 1.5 L to 215 L. For example, the total working volume of each PBR cell may be from 10 L to 200 L, or from 50 L to 150 L. In one example, the total working volume of a PBR cell may be about 100 L.

When two or more PBR cells are interconnected to form a PBR system (see, for example, FIG. 1b), the configuration is preferably such that there is an even number of plastic film tubes at the front as there are at the back of system so that the last plastic film tube in the series of PBR cells may be looped back to be connected to the first plastic film tube in the series of PBR cells to complete the circulation of the PBR system.

When two or more PBR cells are interconnected, preferably the plastic film tubes at the back are placed either directly behind the plastic film tubes in front or at an angle offset to the plastic film tubes in front. Preferably the plastic film tubes are set with a distance of from 50 mm to 300 mm separating back plastic film tubes from front plastic film tubes.

### Temperature Control

The PBR can be thermally regulated either via external means such as a temperature-controlled greenhouse, shade sails or other means of blocking the natural heat of the sun, blankets for retaining heat in the PBR cells during periods of cool weather, or via heating and/or cooling means placed internally in the tubes of the PBR cells or surrounding the PBR cells. If the thermal regulation is internal, preferably it comprises element(s) placed within vertical tubes from the top and connected to a central temperature-controlled heating and chilling unit in order to maintain the cultivation temperature within the PBR. The heating and/or cooling elements may be manufactured from stainless steel.

Preferably the temperature of the PBR cells is maintained from 12° C. to 28° C. The exact temperature can be determined by the skilled reader by reference to the algae being grown. For example, if the algae growing is A. taxiformis, the preferred temperature is from 15° C. to 28° C., for example about 20° C. If the algae growing is A. armata, the preferred temperature is from 12° C. to 23° C., for example about 17° C.

### Light Source

Preferably the PBR cells of the present invention are exposed to a light intensity of from 100 μmols m⁻²·s⁻¹ to 1500 μmols m⁻²·s⁻¹, more preferable 150 μmols m⁻²·s⁻¹ to 1000 μmols m⁻²·s⁻¹.

The light source may be natural light or may be provided by man-made electrical lighting. If is used, preferably the electrical lighting provides wavelengths of from 400 nm to 700 nm, to maximise algal growth.

When two or more PBR cells are interconnected, the plastic tubes at the back are preferably placed either directly behind the tubes in front or at an angle offset to the tubes in front. Offsetting tubes enhances light availability to all tubes and is preferable to maximise growth.

The length of the day (photoperiod) is a robust seasonal signal that eukaryotic algae can use to initiate or complete different developmental programs. Multicellular algae such as the macroalgal red algae (Rhodophyta) have a triphasic (gametophyte, carposporophyte, and tetrasporophyte) life cycle.

The PBR cells of the present invention are preferably exposed to a photoperiod of from 8:16 to 14:10 light: dark cycle, more preferable a 10:14 light: dark cycle. This photoperiod maximises the tetrasporophyte stage of the algal life cycle, more preferably maximising the tetrasporophyte stage of Asparagopsis' life cycle.

### Aeration

In order to grow, the algae within a PBR cell of the present invention must be provided with oxygen and carbon dioxide in an aqueous (i.e., water-based) culture medium. Providing oxygen and carbon dioxide may be achieved by aerating the riser portion of the PBR cell. The aeration provides the additional advantage of lifting and mixing the water within the PBR cell and mixing and dispersing the algae growing within the PBR cell.

Preferably the aeration flow rate is from 1 L/min to 70 L/min. The aeration causes the tetrasporophyte cultures to fragment within at least the riser portion of the PBR cell. If the aeration flow rate is too low, then tetrasporophytes aggregate and may form a single mass, and do not form fragments and the system performance may be compromised. If aeration flow rate is too turbulent, then too much fragmentation may occur and tetrasporophytes may not reach large size. More preferably the aeration flow rate is from 1 L/min to 70 L/min or 1 L/min to 30 L/min; for example about 1 L/min to 10 L/min.

There are four types of bubble regimes present in aeration systems. The ratio or presence/absence of the four types will depend on flow rate but can be further influenced by porous sparger type and pore sizes. At small air flow rates, the system operates in the “bubble flow regime” (small bubbles largely dependent on sparger type and pore size). As the flow rate of the air increases, bubbles coalesce to form large bubbles that drive a “slug” of water up the pipe in the “slug flow regime”. As the airflow continues to increase, the large air bubbles become unstable, resulting in the “churn flow regime”. At large air flows, an “annular flow regime” develops and water flow declines. The highest efficiency and maximum capacity of air and water flow, resulting in maximum airlift operation, maximum algal growth and optimal fragmentation, occurs near the slug flow regime/churn flow regime transition region.

Preferably the aeration is provided by admitting compressed air or via a blower into the riser portion of the PBR cell. The aeration may be achieved by the use of porous spargers (for example air-stones, perforated tubes, or perforated plastic) with pore sizes from 10 to 200 μm. More preferably the pore sizes are from 50 μm to 150 μm, for example about 80 μm. These are cheap and easy to set up, reducing the cost of setup and maintenance of the PBR cell and/or PBR system.

CO₂ enriched air (up to 5% v/v) could be used, if desired, through a CO₂ cylinder and a flow meter connected to the compressed air supply line. The presence of CO₂ can help maintain the pH of the system at desired levels.

### Water Flow

Generally, the water flow rate or velocity is determined and controlled by the aeration flow rate—the movement of the air bubbles coincidentally moves the surrounding water. However, if desired a separate water pump may be attached to the PBR cell or PBR system to assist in water flow control. Preferably the water velocity is from 0.1 m/s to 0.7 m/s.

### Nutrients

Seaweed can be grown on different forms of nitrogen (including NO⁻ and NH⁺ as well as organic forms of nitrogen) and phosphate (including inorganic and organic forms of phosphorous), trace metals (including copper, zinc, manganese, molybdenum, iron, and cobalt), and vitamins (including vitamin B12, biotin, and thiamin).

Suitable nutrient media are known to the skilled reader for algal growth. These include commercially available nutrient media (including Cell-Hi™ algal nutrient medium); nutrient media developed from seawater nutrient media recipes (including F/2 media, Provasoli Enriched Seawater, Enriched Seawater, Erdschreiber's Medium); or organic forms of nutrient media (fish farm effluent, sewerage effluent, animal effluent).

### pH

Preferably the pH of the PBR cell is maintained from pH 7 to pH 9. More preferably the pH is maintained at from pH 7.5 to pH 9.0. For example, the pH of the PBR cell may be maintained at about pH 8.1 to 8.8, being the average pH of seawater.

### Methods for Algal Growth

The present invention provides a method of growing algae comprising the steps of:
i. inoculating a photobioreactor (PBR) cell with algal cells, wherein the PBR cell comprises:
   a. a riser portion in the form of a tube;
   b. a downcomer portion in the form of a tube;
   c. a means of joining the riser portion and the downcomer portion to allow liquid flow;
   d. an aeration means;
ii. maintaining the PBR cells in an aqueous culture medium under conditions of light and aeration that suit the growth of the algal cells.

Preferably the algae is a macroalgae, more preferably the macroalgae is an Asparagopsis spp.

Preferably the tubes of the PBR cells are made from UV-resistant, low density plastic film, with a diameter of from 75 to 300 mm, and/or a length of from 500 to 3000 mm. Preferably the total working volume of each PBR cell is from 1.5 to 215 litres.

Preferably the temperature of the PBR cells is maintained from 12° C. to 28° C. Preferably the aeration rate of the PBR cells is from 1 L/min to 70 L/min. Preferably the PBR cells of the present invention are exposed to a light intensity of from 100 μmols m⁻²·s⁻¹ to 1500 μmols m⁻²·s⁻¹ and a photoperiod of from 8:16 to 14:10 light: dark cycle. Preferably the velocity of the aqueous culture medium (water velocity) is from 0.1 m/s to 0.7 m/s. Preferably the pH of the aqueous culture medium is maintained from pH 7 to pH 9.

The method may comprise the further step of harvesting the algae from the PBR cell, preferably in a continuous way. This may be achieved by the use of a sieve (such as a nylon mesh sieve) or other collecting means, inserted into the top of any of the riser or downcomer portions, preferably the downcomer portions. Such a collecting means can harvest some or all of the biomass as the algae passes the collecting means due to water flow, and thus algal flow, through the riser portion and downcomer portions. This is described further below.

The present invention provides a method of growing algae comprising the steps of:
i. growing algal cells in aqueous culture medium;
ii. removing algal biomass from the culture medium to produce a culture of retained algal cells; and
iii. maintaining the culture of retained algal cells in aqueous culture medium under conditions of light and aeration that suit the growth of the algal cells.

The present invention further provides a method of growing algae comprising the steps of:
i. growing algal cells in aqueous culture medium until the algal cells reach a biomass density of greater than 0.1 g/L;
ii. removing algal biomass from the culture medium to produce a culture of retained algal biomass having:
   a. a biomass density of at least 0.1 g/L; and/or
   b. an average fragment diameter less than the average fragment diameter of the algal cells in the aqueous culture medium prior to removing algal biomass; and
iii. maintaining the culture of retained algal biomass in the aqueous culture medium under conditions of light and aeration that suit the growth of the algal cells.

Preferably the algae is a macroalgae, more preferably the macroalgae is an Asparagopsis spp.

Preferably, the method is performed using a PBR cell as herein described.

Growing the algal cells in aqueous culture medium may be achieved by inoculating algae to an aqueous culture medium and maintaining the inoculated algal cells in aqueous culture medium under conditions of light and aeration that suit the growth of the algal cells to reach the chosen biomass density. The algae which is first inoculated into the aqueous culture medium may be collected from its natural water-dwelling environment and/or grown on a solid culture medium using methods known in the art.

By the phrase “an average fragment diameter less than the average fragment diameter of the algal cells in the aqueous culture medium prior to removing algal biomass”, it is meant that the average diameter size of the fragments may be 1%, 5%, 10%, 15%, 20%, 25%, 30%, 35%, 40%, 45%, 50%, 55%, 60%, 65%, 70%, 75%, 80%, 85%, 90%, or 95% less than the average diameter size of the fragments prior to removing algal biomass. By the phrase “an average fragment diameter more than the average fragment diameter of the algal cells in the aqueous culture medium prior to removing algal biomass”, it is meant that the average diameter size of the fragments may be 1%, 5%, 10%, 15%, 20%, 25%, 30%, 35%, 40%, 45%, 50%, 55%, 60%, 65%, 70%, 75%, 80%, 85%, 90%, or 95% more than the average diameter size of the fragments prior to removing algal biomass.

Preferably the algal cells in aqueous culture medium have a biomass density of greater than 1.0 g/L or 2.0 g/L or 3.0 g/L or 4.0 g/L or 5.0 g/L. Preferably the algal cells in aqueous culture medium have a biomass density of greater than 3.0 g/L. The algal cells in aqueous culture medium may have a biomass density of up to about 13 g/L or 12 g/L or 11 g/L or 10 g/L or 9 g/L or 8 g/L. Expressed as a range, the algal cells in aqueous culture medium may have a biomass density of from greater than 0.1 g/L to 12.0 g/L or from greater than 1.0 g/L to 11.0 g/L or from greater than 2.0 g/L to 10.0 g/L or from greater than 3.0 g/L to 9.0 g/L or from greater than 4.0 g/L to 8.0 g/L or from greater than 5.0 g/L to 7.0 g/L. That said, any minimum and maximum may be combined without limitation; for example, the algal cells in aqueous culture medium may have a biomass density of from greater than 0.1 g/L to 8.0 g/L or from greater than 1.0 g/L to 9.0 g/L or from greater than 4.0 g/L to 7.0 g/L etc. Preferably, the algal cells in aqueous culture medium have a biomass density of from greater than 4.0 g/L to 8.0 g/L.

The algal cells in aqueous culture medium may be comprised of biomass fragments having a diameter of less than 2.0 mm and/or of at least 2.0 mm. Biomass fragments may have a diameter of up to about 12 mm or 11 mm or 10 mm or 9 mm or 8 mm or 7 mm. Expressed as a range, the algal cells in aqueous culture medium may be comprised of biomass fragments having a diameter of from less than 2.0 mm to 12 mm or 11 mm or 10 mm or 9 mm or 8 mm or 7 mm. Preferably the provided algal cells in aqueous culture medium include biomass fragments having a diameter of greater than 2.0 mm. Preferably, about 30% to about 95% or about 40% to about 90% or about 50% to about 85% or about 60% to about 80% of the algal cells in aqueous culture medium is comprised of biomass fragments having a diameter of greater than 2.0 mm. Preferably, the provided algal cells in aqueous culture medium include biomass fragments having a diameter of greater than 4 mm. Preferably, the provided algal cells in aqueous culture medium include biomass fragments having a diameter of greater than 5 mm. Preferably, immediately prior to the removal of algal biomass, the provided algal cells in aqueous culture medium is comprised of biomass fragments having a diameter of up to about 7 mm.

Removal of algal biomass may be referred to herein as “harvesting”.

A culture of algal cells from which algal biomass has been removed may generally be referred to as a “culture of retained algal cells”. Immediately after algal biomass has been removed, the culture of retained algal cells preferably has a biomass density of at least 1.0 g/L or 2.0 g/L or 3.0 g/L or 4.0 g/L. The culture of retained algal cells may have a biomass density of up to about 9.0 g/L or 8.0 g/L or 7.0 g/L or 6.0 g/L. Expressed as a range, the culture of retained algal cells may have a biomass density of from 0.1 g/L to 9.0 g/L or from 1.0 g/L to 8.0 g/L or from 2.0 g/L to 7.0 g/L or from 3.0 g/L to 6.0 g/L. That said, any minimum and maximum may be combined without limitation; for example, the culture of retained algal cells may have a biomass density of from 0.1 g/L to 8.0 g/L or from 1.0 g/L to 6.0 g/L or from 3.0 g/L to 7.0 g/L etc. In preferred embodiments, the culture of retained algal cells has a biomass density of at least 2.0 g/L and preferably of from 2.0 g/L to 5.0 g/L.

Preferably the culture of retained algal cells includes biomass fragments having a diameter of about or less than 2 mm. Preferably, at least about 30% or 40% or 50% or 60% or 70% or 80% of the culture of retained algal cells is comprised of biomass fragments having a diameter of less than 2 mm. Up to about 95% or 90% or 85% or 80% of the culture of retained algal cells may be comprised of biomass fragments having a diameter of less than 2.0 mm. Expressed as a range, about 30% to about 95% or about 40% to about 90% or about 50% to about 85% or about 60% to about 80% of the culture of retained algal cells may be comprised of biomass fragments having a diameter of less than 2 mm. The culture of retained algal cells may also include biomass fragments having a diameter of from 2 mm to 4 mm. Preferably, about 40%, 50%, 60%, 70% or 80%, to about 85%, 90%, 95% or 100%, of the culture of retained algal cells is comprised of biomass fragments having a diameter of about or less than 4 mm.

Methods for determining biomass density are known to those of skill in the art. One method involves taking a sample of algal biomass in a known quantity of aqueous culture medium and drying and weighing the algal biomass to determine its dry weight per volume of aqueous culture medium.

Methods for determining an average fragment diameter are known to those of skill in the art. One method involves taking a whole sample of biomass and measuring the size of biomass fragments using a measuring tool such as a calliper. This may be performed under a microscope or other magnification. It is not necessary to measure every biomass fragment in aqueous culture medium nor even every biomass fragment in a sample. The average may be determined by measuring 20 or more biomass fragments. That said, determining an average fragment diameter is not always necessary. This is because, in order to produce a culture of retained algal cells having an average fragment diameter less than the average fragment diameter of the algal cells in aqueous culture medium prior to removing algal biomass, the harvesting will function by removing algal biomass having an average fragment diameter of greater than the average fragment diameter of the algal cells in aqueous culture medium prior to removing algal biomass. This may be achieved by selectively removing biomass fragments of size greater than the average, or biomass fractions of larger size. In other words, removing medium and large biomass fragments from algal cells in aqueous culture medium which contains very small, small, medium and large biomass fragments, will result in a culture of retained algal cells having an average fragment diameter less than the average fragment diameter of the algal cells in aqueous culture medium prior to removing algal biomass. This may be achieved by the use of a sieve or other collecting means which disproportionately removes biomass fragments of size greater than the average. This is achievable using collecting means having an appropriate pore size. For example, a pore size of 2 mm may be used to remove biomass fragments having a diameter of greater than 2 mm while allowing biomass fragments having a diameter of greater than 2 mm to pass through and remain in the culture of retained algal cells.

Preferably, the removed algal biomass includes biomass fragments having a diameter of about or greater than 2.0 mm (i.e., small, medium and/or large). Preferably, the removed algal biomass includes biomass fragments having a diameter of about or greater than 4.0 mm (i.e., medium and/or large). Biomass fragments in the algal biomass may have a diameter of up to about 12 mm or 11 mm or 10 mm or 9 mm or 8 mm or 7 mm. Expressed as a range, the algal cells in aqueous culture medium may comprise biomass fragments having a diameter of from about or greater than 2.0 mm or 4.0 mm, to 12 mm or 11 mm or 10 mm or 9 mm or 8 mm or 7 mm. Preferably, at least about 60% or 70% or 80% or 90% or 95%, to 100%, of the algal biomass is comprised of biomass fragments having a diameter of about or greater than 2.0 mm or about or greater than 4.0 mm. Preferably, the algal biomass is comprised of biomass fragments having a diameter of up to about 7 mm (i.e., large).

In preferred combinations of the algal cells in aqueous culture medium, the culture of retained algal cells, and the removed biomass:
i. the algal cells in aqueous culture medium have a biomass density of greater than 3.0 g/L, preferably of from greater than 4.0 g/L to 8.0 g/L, and/or about 60% to about 80% of the provided algal cells in aqueous culture medium is comprised of biomass fragments having a diameter of greater than 2.0 mm;
ii. after removal of biomass, the culture of retained algal cells has a biomass density of at least 2.0 g/L and preferably of from 2.0 g/L to 5.0 g/L, and/or about 60% to about 80% of the culture of retained algal cells is comprised of biomass fragments having a diameter of less than 2 mm, and about 70% to about 100% of the culture of retained algal cells is comprised of biomass fragments having a diameter of less than 4.0 mm; and
iii. about 70% to about 95% of the removed algal biomass is comprised of biomass fragments having a diameter of about or greater than 4.0 mm and includes biomass fragments having a diameter of up to about 7.0 mm.

After removal of algal biomass, the culture of retained algal cells is maintained in aqueous culture medium under conditions of light and aeration that suit the growth of the algal cells. The culture of retained algal cells may then be grown into a culture akin to the algal cells in aqueous culture medium.

Preferably the culture of algal cells and/or the culture of retained algal cells are maintained in aqueous culture media at a temperature of from 12° C. to 28° C. Preferably the culture medium is aerated at a rate of from 1 L/min to 70 L/min. Preferably the algal cells are exposed to a light intensity of from 100 μmols m⁻²·s⁻¹ to 1500 μmols m⁻²·s⁻¹ and a photoperiod of from 8:16 to 14:10 light: dark cycle. Preferably the velocity of the aqueous culture medium (water velocity) is between 0.1 m/s to 0.7 m/s. Preferably the pH of the aqueous culture medium is maintained from pH 7 to pH 9.

The culture of retained algal cells may be maintained under growth condition until algae growth results in algal cells in aqueous culture medium having the characteristics described above, and a second removal of algal biomass from the culture medium, having the characteristics as described above, may be performed to produce a second culture of retained algal cells having the characteristics as described above; i.e., each of steps i. to iii. may be repeated using the culture of retained algal cells produced in the previous step ii. Each sequence of steps i. to iii. may be referred to as a “growing cycle”. That is, in preferred embodiments, in each growing cycle the algal cells in aqueous culture medium have a biomass density of preferably from greater than 5.0 g/L to 7.0 g/L, and after removal of algal biomass the culture of retained algal cells has a biomass density of from 3.0 g/L to 5.0 g/L, and the removed algal biomass includes a disproportionate amount of small, medium and/or large biomass fragments.

The method may be performed for 1, 2, 5, 10, 15, 20, 25 or 30 or more growing cycles, or any integer there between, and may be performed indefinitely. Repeat cycles may be referred to as “continuous harvesting”. After a number of cycles, say 1, 2, 5, 10, 15, 20, 25, 30 or more, an intermediate step may be performed whereby the aqueous culture medium is replaced. This may be achieved by removing substantially all algal biomass. A portion thereof may be inoculated to fresh aqueous culture medium. This intermediate step may replace a step ii. and iii. during continuous harvesting. This intermediate step may also be inserted between a step ii. and iii. This will result in harvested algal biomass having an average fragment diameter of less than the average fragment diameter of the algal cells in aqueous culture medium prior to removing algal biomass.

The growth rate of algae in aqueous culture medium begins to slow when the biomass reaches a certain density and/or when biomass fragments reach a certain size. It has been found that by removing biomass fragments of larger size from a culture, a combined advantage may be realized, being to avoid this slowing of growth rate and maintaining a higher growth rate of the smaller fragments, while producing an improved feedstock for ruminant feed comprised in a significant portion by larger size biomass fragments. By producing a culture of retained algal cells having an average fragment diameter less than the average fragment diameter of the algal cells in aqueous culture medium prior to removing algal biomass, harvesting the culture of retained algal cells also allows for the production of a feedstock comprised in a significant portion by smaller size biomass fragments. Smaller size biomass fragments may contain a higher proportion of organically produced metabolites such as cell-wall polysaccharides which may also result in an improved ruminant feed.

### Cultures and Compositions

The present invention thus also provides a culture of retained algal cells being a culture of algal cells from which algal biomass has been removed, said culture of retained algal cells having an average fragment diameter of less than an average fragment diameter of the culture of algal cells immediately prior to removal of the algal biomass.

In producing a culture of retained algal cells having an average fragment diameter of less than an average fragment diameter of the culture of algal cells in aqueous culture medium immediately prior to removal of algal biomass, the removal of biomass will remove algal biomass having an average fragment diameter of greater than the average fragment diameter of the culture of algal cells in aqueous culture medium prior to removing algal biomass. As described above, this may be achieved by the use of a sieve or other collecting means which disproportionately removes biomass fragments of size greater than the average, for example a collecting means having an appropriate pore size.

Preferably, the culture of retained algal cells has a biomass density of at least 1.0 g/L or 2.0 g/L or 3.0 g/L or 4.0 g/L. The culture of retained algal cells may have a biomass density of up to about 9.0 g/L or 8.0 g/L or 7.0 g/L or 6.0 g/L. Expressed as a range, the culture of retained algal cells may have a biomass density of from 0.1 g/L to 9.0 g/L or from 1.0 g/L to 8.0 g/L or from 2.0 g/L to 7.0 g/L or from 3.0 g/L to 6.0 g/L. Any minimum and maximum may be combined without limitation; for example, the culture of retained algal cells may have a biomass density of from 0.1 g/L to 8.0 g/L or from 1.0 g/L to 6.0 g/L or from 3.0 g/L to 7.0 g/L etc. In preferred embodiments, the culture of retained algal cells has a biomass density of at least 2.0 g/L and preferably of from 2.0 g/L to 5.0 g/L.

Preferably the culture of retained algal cells includes biomass fragments having a diameter of about or less than 2 mm. Preferably, about 30% to about 95% or about 40% to about 90% or about 50% to about 85% or about 60% to about 80% of the culture of retained algal cells is comprised of biomass fragments having a diameter of less than 2 mm. The culture of retained algal cells may also include biomass fragments having a diameter of from 2 mm to 4 mm. Preferably, about 40%, 50%, 60%, 70% or 80% to about 90%, 95% or 100% of the culture of retained algal cells is comprised of biomass fragments having a diameter of less than 4 mm.

Preferably, the culture of retained algal cells has a biomass density of from 2.0 g/L to 7 g/L and about 60% to about 80% of the culture of retained algal cells is comprised of biomass fragments having a diameter of less than 2 mm, and about 70% to about 100% of the culture of retained algal cells is comprised of biomass fragments having a diameter of less than 4 mm.

The present invention also provides a composition comprising algal biomass removed from a culture of algal cells in aqueous culture medium, said removed algal biomass having an average fragment diameter of greater than an average fragment diameter of the culture of algal cells in aqueous culture medium from which the algal biomass was removed.

Producing removed algal biomass having an average fragment diameter of greater than an average fragment diameter of a culture of algal cells in aqueous culture medium from which the algal biomass was removed is achievable, as described above, by the use of a sieve or other collecting means which disproportionately removes biomass fragments of size greater than the average, for example a collecting means having an appropriate pore size.

Preferably the removed algal biomass includes biomass fragments having a diameter of about or greater than 2 mm and/or about or greater than 4 mm. Preferably, at least about 60% or 70% or 80% or 90% or 95%, to 100%, of the removed algal biomass is comprised of biomass fragments having a diameter of about or greater than 2.0 mm or about or greater than 4.0 mm. Preferably, the removed algal biomass includes biomass fragments having a diameter of up to about 7 mm.

Preferably, about 70% to about 95% of the removed algal biomass is comprised of biomass fragments having a diameter of about or greater than 4 mm and comprised of biomass fragments having a diameter of up to about 7 mm.

The present invention also provides a composition comprising algal biomass, wherein at least 60%, preferably 70%, 80% or 90% of the algal biomass has a diameter of at least 2 mm. The present invention also provides a composition comprising algal biomass, wherein about 60% to about 80% of the algal biomass is comprised of biomass fragments having a diameter of less than 2 mm. Algal biomass so-characterised may be obtained as described herein.

The culture of retained algal cells may also be harvested. Accordingly, the present invention also provides a composition comprising retained algal biomass removed from a culture of retained algal cells, said culture of retained algal calls being a culture of algal cells from which algal biomass has been removed, said retained algal biomass having an average fragment diameter of less than an average fragment diameter of the culture of algal cells from which algal biomass was removed. Algal biomass harvested from culture of retained algal cells may be generally referred to as “retained algal biomass”.

Producing retained algal biomass having an average fragment diameter of less than an average fragment diameter of a culture of algal cells in aqueous culture medium from which removed biomass was removed is achievable, as described above, by the use of a sieve or other collecting means which disproportionately removes biomass fragments of size greater than the average, and then collecting the retained biomass fragments of size less than the average.

Preferably the retained algal biomass includes biomass fragments having a diameter of less than 2 mm. Preferably, at least about 30% or 40% or 50% or 60% or 70% or 80% of the retained algal biomass is comprised of biomass fragments having a diameter of less than 2 mm. Preferably, about 40%, 50%, 60%, 70% or 80%, to about 85%, 90%, 95% or 100%, of the culture of retained algal cells is comprised of biomass fragments having a diameter of about or less than 4 mm. Preferably, about 60% to about 80% of the retained algal biomass is comprised of biomass fragments having a diameter of less than 2 mm. Preferably, about 70% to about 100% of the retained algal biomass is comprised of biomass fragments having a diameter of less than 4 mm.

The algal biomass described herein (algal biomass, retained algal biomass, removed algal biomass) finds particular utility in ruminant feed. The algal biomass may be admixed with other ingredients suitable for use in ruminant fed.

### General

Those skilled in the art will appreciate that the present invention is susceptible to variations and modifications other than those specifically described. The disclosure includes all such variation and modifications. The disclosure also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the disclosure.

The present invention is not to be limited in scope by any of the specific embodiments described herein. These embodiments are intended for the purpose of exemplification only. Functionally equivalent products, formulations and methods are clearly within the scope of the present invention.

The present invention may include one or more range of values (eg. size, weight, percentage etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers.

It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the disclosure.

Other definitions for selected terms used herein may be found within the detailed description of the present invention and apply throughout. Unless otherwise defined, all other scientific and technical terms used herein have the same meaning as commonly understood to one of ordinary skill in the art to which the disclosure belongs. The term “active agent” may mean one active agent, or may encompass two or more active agents.

The following examples serve to more fully describe the manner of using the above-disclosure, as well as to set forth the best modes contemplated for carrying out various aspects of the disclosure. It is understood that these methods in no way serve to limit the true scope of this disclosure, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the disclosure as set out above.

### Example 1

### Thin Film Photobioreactor Cells (PBR Cells)

An example of a PBR cell of the present invention is provided in FIG. 1.

In FIG. 1, a photobioreactor (PBR) cell [100] of the present invention comprises a riser portion [110] and a downcomer portion [120]. The riser portion [110 ] is interconnected with the downcomer portion [120] by a top H-connector [130] and a bottom U-connector [140].

A source of compressed air (not shown) provides aeration into the PBR cell [100] through aeration tube [150] as evidenced by bubbles [160], to induce an upwards flow of air and water in riser portion [110]. This upwards flow in riser portion [110] then pushes water and air through H-connector [130] into downcomer portion [120]. The water and air then flow through U-connector [140] back into riser portion [110]. The flow of air and water is indicated by the bold arrows.

The algae [160] grow in the riser portion [110] and the downcomer portion [120].

### Example 2

### Algal Growth in PBR System

### Methods

PBR cells comprising of two connected vertical plastic film tubes were interconnected in series, with one tube of each PBR cell acting as the riser portion and the second tube of each PBR cell as the downcomer portion alternately, to form a PBR system.

The plastic film tubes were made from UV-resistant low-density polyethylene (LDPE) plastic tubing of 100 microns density. Each plastic film tube had a diameter of 150 mm and a height of 2500 mm. The plastic film tubes were interconnected with polyvinylchloride (PVC) bottom U-connectors and top H-connectors (FIG. 2a). The U-connectors and H-connectors were clamped to the plastic film via a rubber coupling and stainless steel (316) ring clamp to ensure no slippage of the plastic film from the U-connector or H-connector and provide a water-tight seal.

The H-connectors were open at the top to allow off-gassing from the aeration.

In the PBR system, each PBR cells' riser portion and downcomer portion formed a vertical loop, with the vertical tubes spaced 150 mm apart. The total working volume of each PBR cell was 100 L (each cell has a riser portion and downcomer portion, plus 8 L of volume in the U-connectors and 4 litres of volume in the H-connectors). As shown in FIG. 2b, multiple cells were interconnected and the configuration was such that there was an even number of tubes at the front as there were at the back of system, so that the last tube in the series of cells looped back to be connected to the first tube in the series of cells to complete the circulation of the system.

In the PBR system, the tubes at the back were placed at an angle offset to the tubes in front at a distance of 90 mm, with 150 mm separating back tubes from front tubes.

An aeration flow rate of 20-30 L/min was used, administered using porous spargers (air-stones) with pore sizes between 80 μm and air-bubble sizes between 3-5 mm in diameter.

The PBR was thermally regulated externally via setting the PBR system up in a temperature-controlled room to maintain the 20° C. within the PBR system.

The algal cultures were maintained by feeding with nitrogen (including NO⁻ and NH⁺ as well as organic nitrogen) and phosphate (including inorganic and organic phosphate), trace metals (including copper, zinc, manganese, molybdenum, iron, and cobalt), and vitamins (including vitamin B12, biotin, and thiamin).

A seaweed harvesting sieve made from nylon mesh was deployed that could harvest biomass when dipped into the top of any of the downcomer tubes. The nylon mesh harvesting sieve had a pore size of less than 0.5 mm and could harvest biomass completely. Other seaweed harvesting sieves with different pore sizes could partially harvest biomass, depending on the pore size of the sieve. A sieve with a pore size of 3.55 mm could collect 95% or more of the large biomass having a diameter of >6 mm. A stainless mesh sieve with a pore size of 2.88 mm could collect 95% or more of the large biomass, and 90% or more of the medium biomass having a diameter of 4-6 mm. A stainless mesh sieve with pore size of 2.00 mm could collect 95% or more of the large biomass, 90% or more of the medium biomass, and 40% or more of the small biomass having a diameter of 2-4 mm. Sieves could also be made out of stainless-steel mesh.

### Results

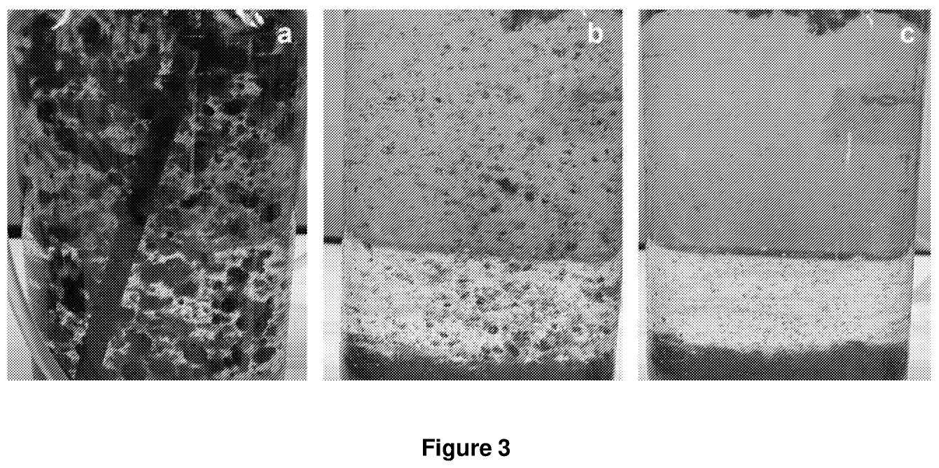

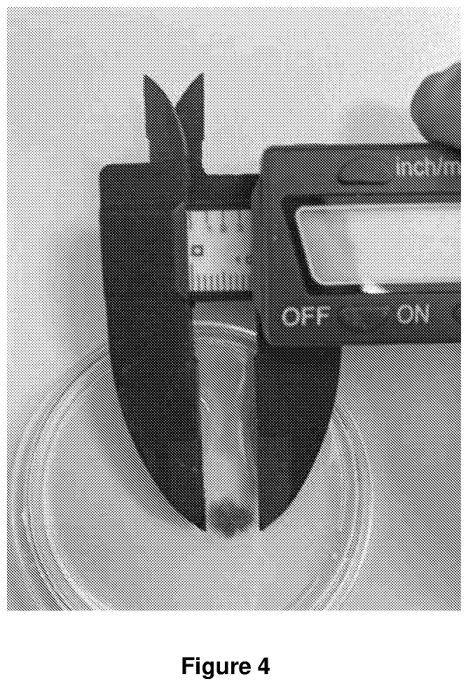

Using the PBR system described above, tetrasporophyte cultures of Asparagopsis spp. were maintained at a culture density of from 3-7 g/L. The airlift mechanism, aeration flow rate and bubble size caused the tetrasporophyte cultures to naturally self-fragment within the riser portion of the PBR cells and continue to grow so that a continuous harvest process was required to maintain culture density. For example, within a dense culture of 5 g/L there were a range of tetrasporophyte sizes, with harvestable size of >8 mm in diameter. The culture was also made up of medium (3-7 mm) fragments and small (<3 mm) fragments (FIG. 3). Optimized operation of the photobioreactor cell assembly demonstrated a 30 day production cycle from 0.5 mm fragments to harvestable size of 10 mm for tetrasporophytes (FIG. 4) when grown at a light intensity of from 150 to 1000 μmols m⁻²·s⁻¹ and photoperiod of 10:14 light: dark cycle.

Tetrasporophyte cultures of Asparagopsis spp. maintained at 5 g/L wet weight achieved an average growth rate of between 10-20% biomass per day. This implies the need for a 0.5-1 g/L wet weight biomass per day continuous harvest to maintain culture density at 5 g/L.

By extrapolation, a 1000 litre PBR system (ten interconnected 2500 mm×150 mm PBR cells) could supply from 500 to 1000 g wet weight per day or 182.5 to 365 kg wet weight per year under natural light intensities of 150 to 1500 μmol m⁻²·s⁻¹. A 1-hectare site could house 1100 by 1000 litre PBR cell modules (with 1 m perimeter to allow adequate access to light and for manoeuvrability around each PBR system) and produce 200 to 400 tonnes of Asparagopsis biomass per annum through year-round sustained and continuous culture.

### Example 3

### Algal Growth and Biomass Removal in PBR System

Asparagopsis taxiformis seaweed juveniles were clonally produced from the fragmentation of mature tetrasporophytes. Fragmentation was achieved by macerating tetrasporophytes in 50 ml of seawater. Maceration was achieved by using a milk frother, stick mixer or blender, which chops the tetrasporophytes into thousands of fragments within 20-30 seconds. The fragments range in size from 0.3-1.5 mm in length of maximum long axis direction.

Fragments with a total biomass of 80 g fresh weight were inoculated into a PBR system similar to that described above but expanded in series to a 1000 L multi-tubular airlift photobioreactor system. The photobioreactor system was set up on appropriate racking indoors in controlled environmental conditions and filled with filtered seawater.

Each riser portion tube in the PBR system series was aerated at its base using compressed air fed through an air stone (upwelling tube) whilst every alternate downcomer portion tube in the series acted as a downwelling tube. Aeration was set at an average of 25 L/min to provide sufficient lift for the fragments and to circulate water around the PBR system. After 30 days of culture, the density of a culture was more that 5 g/L.

### Results

On three separate days, the average daily growth rate using the PBR system was estimated to be 15%±5%. This average daily growth rate of 15% was achieved using suitable condition of temperature, light and supply of nutrient. The light intensity was set as 1000 μmols m⁻²·s⁻¹ during the maintenance of the culture at 5 g/L.

Using the PBR system described above, tetrasporophyte cultures of Asparagopsis spp. were maintained at culture density of 5 g/L. The culture density was maintained by continuous daily harvesting. The amount to harvest daily was calculated based on the average daily growth rate of 15%.

The culture was made up of large biomass fragments (>6 mm), medium biomass fragments (4-6 mm), small biomass fragments (2-4 mm) and very small biomass fragments (<2 mm). Stainless mesh sieves with pore size of 2.00 mm were used for harvest algal biomass by setting them on top of the downcomer tubes. The stainless mesh sieve allows sizes of tetrasporophytes less than the pore size to pass through while collecting tetrasporophytes of size greater than the pore size. The stainless mesh sieve with pore size of 2.00 mm collects at least 60% of the small, medium and large tetrasporophytes, while leaving behind at least 70% of the retained very small biomass fragments in the PBR system.

After a week's maintenance, the whole biomass was harvested and weighed which revealed that the maintenance was successful.

Thus, the maintenance of a culture of density of 3-7 g/L and a daily growth rate of more than 10% was achieved by continuous daily harvesting of the algal culture in the present PBR system. The daily harvesting process could produce a feedstock made of Asparagopsis tetrasporophytes comprising more than 85% of the medium fragments and large fragments.

1. A photobioreactor (PBR) cell comprising:
a) a riser portion in the form of a tube;
b) a downcomer portion in the form of a tube;
c) a means of joining the riser portion and the downcomer portion to allow liquid flow;
d) an aeration means.

2. The PBR cell of claim 1 wherein the tubes of the PBR cells:
i. are made from UV-resistant, low density plastic film;
ii. have a diameter of from 75 to 300 mm;
iii. have a length of from 500 to 3000 mm; and/or
iv. have a total working volume of from 1.5 to 215 litres.

3. A method of growing algae comprising the steps of:
i. inoculating a photobioreactor (PBR) cell with algal cells, wherein the PBR cell comprises:
a. a riser portion in the form of a tube;
b. a downcomer portion in the form of a tube;
c. a means of joining the riser portion and the downcomer portion to allow liquid flow;
d. an aeration means;
ii. maintaining the PBR cells in an aqueous culture medium under conditions of light and aeration that suit the growth of the algal cells.

4. The method of claim 4 wherein the algae is an Asparagopsis spp.

5. The method of claim 3 wherein:
i. the temperature of the PBR cells is maintained from 12° C. to 28° C.;
ii. the aeration rate of the PBR cells is from 1 to 70 L/min;
iii. the PBR cells are exposed to a light intensity of from 150 to 1500 μmols m⁻²·s⁻¹; and/or
iv. the photoperiod the PBR cells are exposed to is from 8:16 to 14:10 light: dark cycle.

6. A method of growing algae comprising the steps of:
i. providing algal cells in aqueous culture medium having a biomass density of greater than 0.1 g/L and an average fragment diameter;
ii. removing algal biomass from the culture medium to produce a culture of retained algal cells having:
a. a biomass density of at least 0.1 g/L and
b. an average fragment diameter less than the average fragment diameter of the algal cells in aqueous culture medium prior to removing algal biomass; and
iii. maintaining the culture of retained algal cells in aqueous culture medium under conditions of light and aeration that suit the growth of the algal cells.

7. The method of claim 6, wherein the algal cells in aqueous culture medium have a biomass density of from greater than 1.0 g/L to 11.0 g/L.

8. The method of claim 6, wherein the algal cells in aqueous culture medium is comprised of biomass fragments having a diameter of from less than 2.0 mm to 12 mm.

9. The method of claim 6, wherein about 60% to about 80% of the algal cells in aqueous culture medium is comprised of biomass fragments having a diameter of greater than 2.0 mm.

10. The method of claim 6, wherein the culture of retained algal cells has a biomass density of from 1.0 g/L to 8.0 g/L.

11. The method of claim 6, wherein the culture of retained algal cells is comprised of biomass fragments having a diameter of about or less than 2 mm.

12. The method of claim 6, wherein about 60% to about 80% of the culture of retained algal cells is comprised of biomass fragments having a diameter of less than 2 mm.

13. The method of claim 6, wherein the removed algal biomass is comprised of algal biomass having an average diameter of about or greater than 2 mm, and of up to about 7 mm.

14. The method of claim 6, wherein at least 60% of the algal biomass has a diameter of at least 2 mm.

15. The method of claim 6, wherein the algal cells in aqueous culture medium have a biomass density of greater than 2.0 g/L, and removing algal biomass from the culture medium produces a culture of retained algal cells having a biomass density of greater than 2.0 g/L.

16. A culture of algal cells produced using a PBR cell of claim 1.

17. A culture of retained algal cells being a culture of algal cells in aqueous culture medium from which algal biomass has been removed, said culture of retained algal cells having an average fragment diameter of less than an average fragment diameter of the culture of algal cells immediately prior to removal of the algal biomass.

18. A composition comprising algal biomass removed from a culture of algal cells in aqueous culture medium, said algal biomass having an average fragment diameter of greater than an average fragment diameter of the culture of algal cells from which the algal biomass was removed.

19. The composition of claim 18, wherein at least 60% of the algal biomass has a diameter of at least 2 mm.

20. A composition comprising algal biomass, wherein at least 60% of the algal biomass has a diameter of at least 2 mm.

21. A composition comprising algal biomass removed from the culture of retained algal cells of claim 17.

22. A composition comprising retained algal biomass removed from a culture of retained algal cells, said culture of retained algal calls being a culture of algal cells from which algal biomass has been removed, said retained algal biomass having an average fragment diameter of less than an average fragment diameter of the culture of algal cells from which algal biomass was removed.

23. The composition of claim 22, wherein about 60% to about 80% of the retained algal biomass is comprised of biomass fragments having a diameter of less than 2 mm.

24. A composition comprising algal biomass, wherein about 60% to about 80% of the algal biomass is comprised of biomass fragments having a diameter of less than 2 mm.

25. A culture of retained algal cells produced by the method of claim 6.
